(19) United States
(12) Patent Application Publication
(10) Pub. No.: US 2006/0241175 A1
(43) Pub. Date: Oct. 26, 2006

(54) Vehicle for topical delivery of anti-inflammatory compounds

(76) Inventors: Joseph Schwarz, Richmond Hill (CA); Michael Weisspapir, Toronto (CA)

Correspondence Address: MCCARTHY TETRAULT LLP, BOX 48, SUITE 4700,, 66WELLINGTON STREET WEST, TORONTO, ON, M5K 1E6, CA

(21) Appl. No.: 11/454,921

(22) Filed: Jun. 19, 2006

Related U.S. Application Data
(63) Continuation-in-part of application No. 11/080,812, filed on Mar. 16, 2005.
Application No. 11/080,812 is a continuation-in-part of application No. 10/255,951, filed on Sep. 27, 2002.

(51) Int. Cl. A61K 31/355; A61K 31/125; A61K 31/045

(52) U.S. Cl. 514/458; 514/692; 514/729

(57) ABSTRACT

A vehicle for topical delivery which contains a liquid eutectic mixture of hydrophobic compounds.

### CROSS-REFERENCE TO RELATED APPLICATIONS

This application is a continuation-in-part application of U.S. patent application Ser. No. 11/080,812 filed Mar. 16, 2005, which in turn is a continuation-in-part application of U.S. patent application Ser. No. 10/255,951 filed Sep. 27, 2002.

### FIELD OF THE INVENTION

The present invention relates to the preparation of semisolid formulations for topical delivery of pharmaceutically active ingredients, designed for pain control and inflammation treatment.

### BACKGROUND OF THE INVENTION

Topical pharmaceutical preparations of different types have been used for treatment of rheumatic and arthritic pain for decades. Semisolid compositions comprise plant derivatives, such as capsaicin (red hot pepper stinging substance) or turpentine (pine tar component) ointments, homeopathic extract and liniments (Opodeldoc Rus), mustard plasters, menthol rubs, essential oil balms and many others were used for a long time, mainly as local irritants. Such irritation improves local blood flow, accelerates injured tissue recovery, and switches attention from chronic pain from inflammation.

By including non-steroid anti-inflammatory drugs (NSAIDs) into ointment or cream application onto the desired location allows for effective control of muscle and joint pain intensity. Moreover, when NSAIDs are applied topically, local drug concentration in muscle and joint tissues is significantly higher than in non-treated sites. Additionally, there is no intensive metabolism in liver (so called “first-pass effect”) because such drugs do not pass through the liver before action.

The required amount of NSAIDs is lower than an oral dose to achieve similar anti-inflammative and analgesic effects. The most common side effect of NSAIDs is serious irritation of stomach and gastro-intestinal mucosa. This is substantially diminished with local topical applications.

Topical NSAID formulations are very popular in Europe, Asia and Far East regions. Examples of compositions include Voltaren Emulgel® (Voltarol™ in UK), a 1.16% Diclofenac diethylammonium emulsion cream with isopropyl alcohol, Feldene® Gel (0.5% Piroxicam water-ethyl alcohol gel), Ibuprofen and Ketoprofen gels of different strengths (5-10%), and 1-10% Indomethacin in alcohol. DMSO-containing creams and many other formulations are widespread in many countries as OTC remedies for muscle pain, sport minor injuries, rheumatic and back pain treatment, etc.

Generally, topical NSAID preparations do not have attributable side-effects such as gastric irritation and internal bleeding. Advantageously, the compounds provide relatively fast action onset and moderate efficacy in treatment of local muscle and joint pain. The main problems of these products is low drug loading due to low solubility in the cream components. High loading can be reached by use of concentrated alcohols, i.e. ethyl alcohol, isopropyl alcohol with polyethylene glycol and propylene glycol suitable as solvents for NSAIDs. Drug loading is high and can easily reach 5-10% or greater, e.g., 5% Ibuprofen gel with isopropyl alcohol, 1% Indomethacin gel based on ethyl alcohol or even 10% Indomethacin ointment with dimethylsulfoxide.

These solvents are widely used for gel preparation, but widespread use is often limited due to the proclivity for skin irritation. A further limitation is realized in fast termination of action for gel preparations since the drug precipitates from solution subsequent to water absorption from the body tissue. Further, solvents in high concentration often irritate the skin due to drying and delipidisation and may initiate contact dermatitis and allergy. Drug, insoluble in water media and body fluids, precipitates in the upper skin layers and does not penetrate inside, seriously limiting anti-inflammatory action. Similar behavior was observed for polyethylene glycol (mixture of PEG-4000 and PEG-400) hydrophilic topical base.

Traditional hydrophobic vehicles such as fixed oils, mineral oil, petrolatum, lanolin and wax based ointments, along with emulsion creams (either O/W or W/O type) are less irritating to human skin, but these present another complication—solubility. Drug loading in such vehicles is limited by the solubility of the drug in the lipid phase. For example, the solubility of Indomethacin in olive or corn oil is below 0.2%, whereas Ketoprofen is about 1.5% and Piroxicam below 0.05%. According to Benita et at. “Submicron Emulsions as Colloidal Drug Carriers for Intravenous Administration: Comprehensive Physicochemical Characterization”, J. Pharm Sci., 1993, Nov. 82 (11), pp. 1069-79, even for low drug loading, stability of the dispersed system is questionable. A 0.1% Indomethacin submicron emulsion lost stability after 1 month storage.

Use of more polar hydrophobic compounds may help to improve solubility of NSAIDs. Tocopherol acetate, triethyl citrate, glycerin monolaurate, glycerin monooleate (Myverol™ 18-9) dissolve between 1.5 and 2 times more Indomethacin or Diclofenac (in acidic form). Nevertheless, this loading is insufficient to obtain an effective NSAID emulsion. Transdermal adhesive systems such as skin patches and plasters with Indomethacin or Diclofenac present low efficacy by the same reasoning.

A further method to increase drug solubility in the oil phase is to use highly polar compounds, miscible with named phase. Solvents such as Ethoxyethylene glycol (Transcutol™), dimethylisosorbide (DMIS), Isopropylideneglycerin (Solketal™), ethoxylated furanyl alcohol (Glucofurol™) visibly boost drug implementing in the separate hydrophobic phase. However, upon mixing with water, most of the solvent is extracted into the water and the dissolved drug precipitates immediately and almost entirely from the oil phase.

Recently developed submicron emulsions (SME) employed as a base for NSAIDs, provides very effective delivery and exert pronounced improvement for drug action in Friedman et al. (U.S. Pat. No. 6,113,921). However, low solubility of NSAIDs in a lipid phase of such emulsions leads to shortened periods of efficacy and drug precipitation from the oil phase during storage. High loading, desirable for optimal activity of topical NSAID preparation for SME is achievable only for highly lipophilic compounds, such as Naproxen, Ketoprofen or Ibuprofen with significantly lower anti-inflammatory activity.

Eutectic mixture use in topical applications is rather limited. An example is EMLA cream, developed by Astra-Zeneca. The liquid, formed by mixing two crystalline bases of local anesthetics, Lidocain and Prilocain due to eutectic formation serves as an oil phase in the cream for topical application. The cream, containing 5% of such oil phase, provides excellent stability and anesthetic action.

In view of the limitation in the anti-inflammatory drug art, there exists a need for an improved composition which overcomes the shortcomings presently encountered.

### SUMMARY OF THE INVENTION

It has been found that a eutectic mixture of camphor, menthol, thymol and similar compounds is a powerful solvent for non-steroidal anti-inflammatory drugs and other substances. The solubility of Indomethacin, Diclofenac, or Ketoprofen in the mixture increased between 3 and 20 fold. As a particular advantage, the eutectic mixture was found to be safe, non-toxic and present synergistic behavior in anti-inflammatory action of NSAIDs due to anti-inflammatory properties of camphor and skin penetration enhancing properties of menthol.

In one embodiment, the invention provides a method for solubilizing a non-steroidal anti-inflammatory drug for topical application, comprising: combining the non-steroidal anti-inflammatory drug with a solvent vehicle comprising a liquid eutectic mixture of at least two hydrophobic compounds selected from the group consisting of camphor, menthol and thymol.

In another embodiment, the invention provides a composition for topical application comprising: a non-steroidal anti-inflammatory drug and a solvent vehicle for solubilizing said non-steroidal anti-inflammatory drug, said solvent vehicle comprising a liquid eutectic mixture of at least two hydrophobic compounds selected from the group consisting of: camphor, menthol and thymol.

In one embodiment, the solvent vehicle further comprises a liquid hydrophobic component such as, pharmaceutically acceptable glycerin ester, aliphatic esters, aromatic esters, waxes, lipids, fats, lipid soluble vitamins, hydrocarbons, silicone polymers, tocopherols, or mixture thereof. In another embodiment, the hydrophobic component is tocopherol, for instances such as alpha-tocopherol or tocopherol acetate.

In one embodiment, the NSAID is selected from the group consisting of indomethacin, diclofenac, ketorolac, piroxicam, tenoxicam, ketoprofen, flurbiprofen, ibuprofen, Naproxen, meloxicam and salicylic acid. In another embodiment the NSAID is slected from the group consisting of diclofenac, piroxicam, indomethacin, ketoprofen, meloxicam, and ibuprofen.

In one embodiment the composition and/or the composition used in the method of the invention further comprises a glyceride (e.g. an acetylated or polar acetylated di or mono-glyceride) and/or, a hydrobobic ester. In one embodiment a hexadecyloctanoate.

In one embodiment the composition and/or the composition used in the method of the invention further comprises a polar triglyceride. For instance, in one embodiment a polar acetylated glyceride, a medium chain triglyceride, or an acetylated monoglyceride.

In one embodiment, the solvent vehicle is evenly distributed in water containing media forming an homogenous mixture. In one embodiment the composition further comprises viscosity modifiers, surfactants, preservatives, fragances and other excipients such as those that can promote stable formulations suitable for topical application to the skin.

The eutectic mixture can be combined with pharmaceutically acceptable oils and lipids and included into topical formulations. The compositions were found to allow much higher drug loading than existing ointment bases and creams, showed no skin irritation and provided enhanced delivery properties for incorporated drugs.

Prior to enhancing on a discussion of the preparation, some general properties of the menthol and camphor will be established.

The menthol used was (1R,2S,5R)-5-methyl-2-(1-methyethyl)-cyclohexanol with a molecular weight of 156.27 and melting point of 42° C. Menthol generally has a peppermint odor. It is well known as a skin irritant and penetration enhancer in Tsuk (U.S. Pat. No. 4,933,184). It is widely used in many topical formulations for relief of arthritic and rheumatic pain. Natural L-menthol exerts a cooling or refreshing sensation due to direct interaction with cold sensitive receptors in the skin. This was established in the Handbook of Pharmaceutical Excipients, Third Edition, ed. A. H. Kibbe, Pharmaceutical Press, London, U.K., 2000, pp. 334-335. Menthol has been used as mild local anesthetic and as volatile aromatic component for breath relief in obstruction and cold treatment in Hughes et al., (U.S. Pat. No. 5,322,689). In one embodiment, the menthol used in the eutectic mixture is selected from the group consisting of D-isomer, L-isomer, racemic menthol or a mixture of menthol isomers.

Similar properties are known for camphor 1,7,7-trimethylbicyclo[2,2,1]heptanone-2, having a molecular weight of 152.24. Camphor has a high melting point (180° C.) and is a very volatile substance with strong pine-like odor that sublimes even at room temperature and pressure. Initially, camphor found use as a stimulant, but now camphor is mainly used as a component in topical preparations. It is often used in nasal decongestants and aromatic compositions. In one embodiment, the camphor used in the eutectic mixture is selected from the group consisting of D-isomer, L-isomer, racemic camphor or a mixture of camphor isomers.

Either menthol or camphor separately or in combination are widely used in topical formulations, mainly due to their irritant action, receptor interaction and specific traditional odor, frequently associated with time-honored remedies. Ben Gay™ ointment, Tiger™ balm, Menthol Chest Rub and similar compositions are well known and popular.

Certain external analgesic products containing between 10% to 60% methyl salicylate, 3% to 11% camphor and 1.25% to 16% menthol, either singly or in combination, cause irritation or mild inflammation of the skin for the purpose of relieving pain in muscles, joints, or viscera distal to the site of application by stimulating depressing cutaneous sensory receptors in Ivy et al. (U.S. Pat. No. 5,013,726).

Topical preparations for joint relief include that provided in Lang et al. (U.S. Pat. No. 4,731,200) for an aqueous-alcohol composition containing benzylidene-camphor derivatives, Ivy et al. (U.S. Pat. No. 5,013,726) for a lotion containing methyl salicylate, camphor and menthol, Ivy et al. (U.S. Pat. No. 5,124,320) for an analgesic lotion containing menthol and camphor, Heywang et al. (U.S. Pat. No. 5,144,081) for a pharmaceutical composition containing camphor and Singh (U.S. Pat. No. 5,175,152) for a composition with methyl salicylate, menthol and camphor.

These substances have been advertised for use in relieving joint pain, such as the elbow, knee, thumb area, ankle, neck, wrist, hand and finger, shoulder, etc.

To improve solubility of non-steroidal anti-inflammatory drugs, a complex mixture of surfactants, polyglycol(s) and glycerides has been used in combination with polymers and sodium or potassium hydroxide solutions as established in Morton et al. (U.S. Pat. No. 5,376,688).

In Kaplun-Fischoff et al., “Testosterone Skin Permeation Enhancement by Menthol Through Formation of Eutectic with Drug and Interaction with Skin Lipids”, J. Pharm Sci. 1997, December, 86 (12) pp. 1394-9, the researchers observed that menthol forms a eutectic mixture with crystalline testosterone. The formed mixture is not liquid, but the composition demonstrated a significant improvement in transdermal penetration of testosterone. According Kaplun-Frischoff et al., menthol affects skin permeation by a dual mechanism: by forming a eutectic with the penetrating compound, thereby increasing its solubility in skin ceramides and by altering the barrier properties of the stratum corneum.

A careful investigation of existing compositions containing menthol and camphor in different ratios showed that there is no one example of specific use of menthol and camphor in combination as eutectic mixture in order to improve solubility of an included drug. All anti inflammatory components used in such formulations are liquid (methylsalycilate, benzylnicotinate, etc.) and easily miscible with oil components of the creams or ointments. There is no limitation for solubility, and these topicals can contain up to 60% of active component, e.g., methylsalycilate), Altadonna (U.S. Pat. No. 5,853,768).

In the documentation there has not been a recognition of a menthol-to-camphor ratio in the eutectic region. In all cases, the existing preparations are used only due to their mild irritative or anti-inflammatory activity (camphor, nicotinic acid derivatives) or skin penetration enhancement properties of menthol itself.

It has now been recognized that a radical increase of drug solubility in a eutectic mixture of polar hydrophobic compounds allows preparation of effective and safe topical formulations with these drugs for external application.

Additional aspects and advantages of the present invention will be apparent in view of the description which follows. It should be understood, however, that the detailed description and the specific examples, while indicating preferred embodiments of the invention, are given by way of illustration only, since various changes and modifications within the spirit and scope of the invention will become apparent to those skilled in the art from this detailed description.

Having thus described the invention, reference will now be made to the accompanying drawings illustrating preferred embodiments.

### BRIEF DESCRIPTION OF THE FIGURES

FIG. 1 is a graphical representation of Diclofenac (free acid) solubility in a mixture of MCT and a menthol/camphor mixture;

FIG. 2 is a graphical representation of Indomethacin solubility in oleaginous vehicles and in a menthol/camphor vehicle;

FIG. 3 is a graphical representation of Piroxicam solubility in oleaginous vehicles and in a eutectic vehicle;

FIG. 4 is a graphical representation of drug content change during storage;

FIG. 5 is a graphical representation of comparative anti-inflammatory activity of topical formulations; and

FIG. 6 illustrates the comparative anti-inflammatory activity of NSAIDS in different topical formulations.

FIG. 7 illustrates the size distribution of particles in non-diluted sample of Example 7.

Similar numerals in the figures denote similar elements.

### DETAILED DESCRIPTION OF THE INVENTION

A mixture of equimolar amounts of crystalline camphor and menthol at room temperature immediately led to liquefied crystals. This mixture was used in the preparations as an effective solvent for some NSAID compounds.

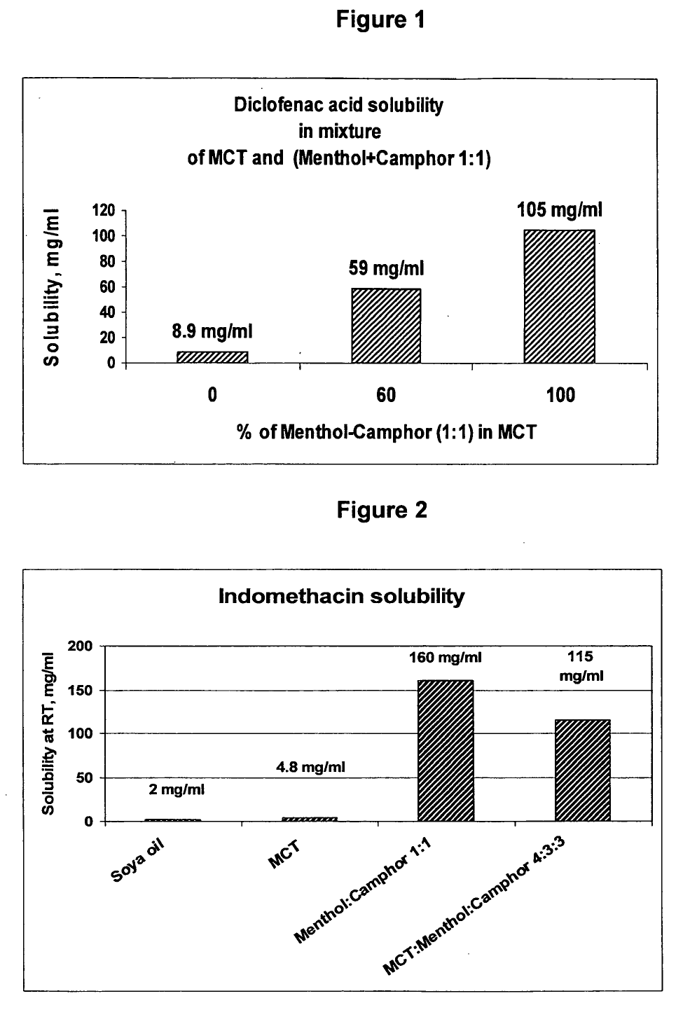

FIG. 1 graphically represents the solubility of Diclofenac (as free acid) in mixtures of medium chain triglycerides (MCT, standard oil vehicle, Labrafac® TGCC) with different levels of added menthol-camphor eutectic mixture. Diclofenac saturation concentration at 25° C. was evaluated by HPLC. Solubility in a pure equimolar menthol-camphor eutectic mixture was found to be 11.8 times higher than in pure MCT.

Similar behavior was observed also for Indomethacin, illustrated graphically in FIG. 2, maximum solubility in equimolar eutectic menthol-camphor mixture is 160 mg/ml, compared with 2 mg/ml in the soy oil or 4.8 mg/ml in MCT oil. For comparison, Ho et. al. “Penetration Enhancement by Menthol Combined with a Solubilization Effect in a Mixed Solvent System”, J. Controlled Release 1998, Feb. 12; 51 (2-3), pp. 301-11, investigated influence of menthol addition (up to 12% by weight) as solubility enhancer for Indomethacin in different pharmaceutical vehicles such as water, ethanol, propylene glycol and their combinations. In any case the maximum solubility hardly reached 2% (approximately 20 mg/ml).

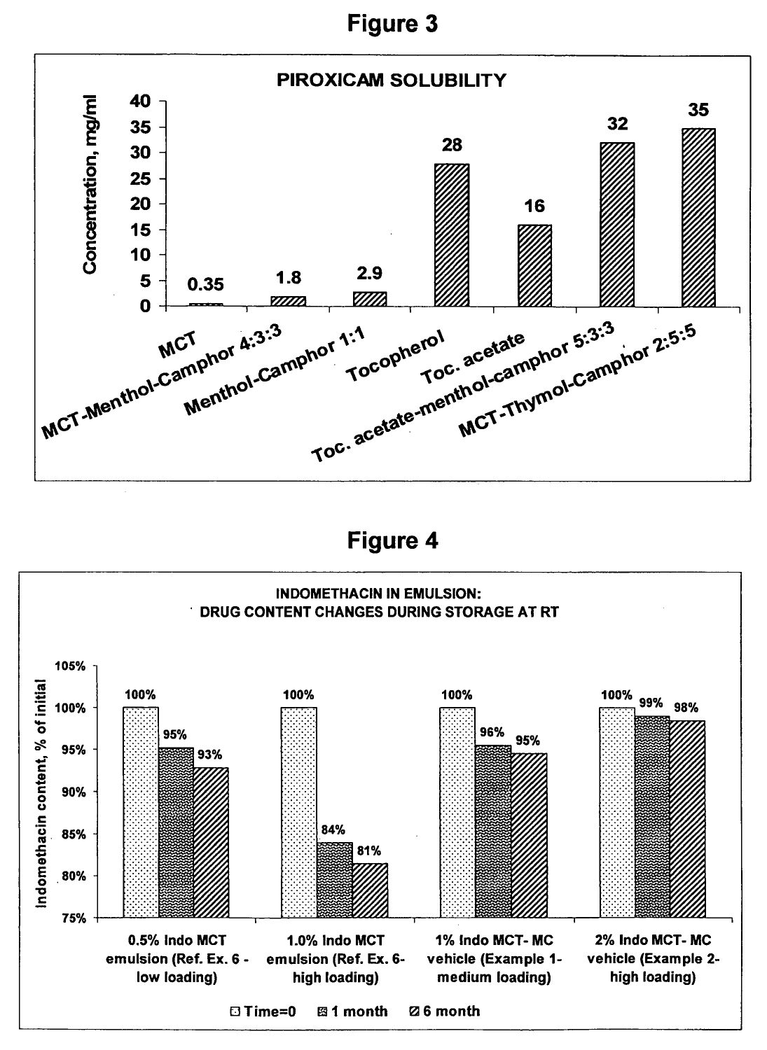

In FIG. 3, further graphic data are presented for Piroxicam.

Piroxicam solubility is significantly lower than aromatic NSAIDs, however, use of the eutectic menthol-camphor mixture increased drug solubility at room temperature between 8 and 11 times, from 0.35 mg/ml in MCT to 2.9-3.2 mg/ml in pure eutectic mixture and to 1.8 mg/ml in MCT with 60% menthol-camphor (1:1) content.

If alpha-tocopherol or tocopherol acetate is used as the oil phase, solubility can reach 30-35 mg/ml for tocopherol-menthol-camphor composition 5:3:3 (parts by weight).

Use of other ratios for menthol-camphor eutectic mixture (e.g., 2:1 or/to 1:2; 3:4 or/to 4:3, 4:1 or/to 1:4) also improves solubility for most of investigated substances but in slightly lower extent. Very significant improvement in solubility was achieved with replacement of menthol for another eutectic forming substance, thymol (2-isopropyl-5-methylphenol, thyme oil component).

Obtained solutions of NSAIDs in lipid phase containing menthol-camphor or another eutectic mixture vehicle are stable in wide temperature range and non-irritating for human and animal skin (Dreize' test). Based on these observations different topical formulations with NSAIDs were prepared and will now be discussed in the examples.

### EXAMPLES

### Example 1

### Indomethacin 1% Cream

TABLE 1

| | | Per 250 |
|---|---|---|
| CREAM INGREDIENTS | % | g cream |
| Indomethacin USP | 1.00 | 2.5 |
| Medium Chain Triglycerides (Labrafac ® CCTG) | 4.00 | 10 |
| Soy Lecithin (Phospholipon ® S-80) | 1.00 | 2.5 |
| (±) Camphor USP | 3.00 | 7.5 |
| L-(−)-Menthol USP | 3.00 | 7.5 |
| Tween ™-80 (Polysorbate 80, USP) | 1.60 | 4.0 |
| TPGS (Tocopherol polyethylene glycol 1000 succinate) | 0.80 | 2.0 |
| Sodium Ethylenediamine tetraacetate (EDTA sodium) | 0.10 | 0.25 |
| Carbopol ® 971 P | 1.50 | 3.75 |
| Glycerin USP | 2.50 | 6.25 |
| Water | 81.50 | 203.75 |

Vehicle (Eutectic Mixture) Preparation:

(±) Camphor and L-Menthol were mixed together during heating at between 40 and 50° C. until a clear liquid was obtained.

Oil Phase Preparation:

Soy lecithin, MCT oil and TPGS were mixed together at 45° C. until a homogenous solution was obtained. Tween™-80 as then added, followed by the addition of the eutectic mixture vehicle. The mixture was stirred until completely dissolved. Indomethacin (USP grade) was added to the warm mixture and stirred for 10 minutes at 45° C. until completely dissolved.

Water Phase Preparation:

EDTA disodium salt, glycerin and Tween™-80 were added to water (90% of calculated amount) and stirred until completely dissolved.

Emulsification:

The solution was combined with the oil phase, mixed thoroughly using appropriate mixer and homogenized using high pressure homogenizer (Avestin® C-5) at 8,000-12,0009 psi, (600-800 bar). The mixture was passed through the homogenizer between 2 and 3 times.

Cream Preparation:

In a separate vessel Carbopol® 971 P was mixed with 10% of calculated amount of water and soaked for between 2 and 6 hours. Carbopol® paste was combined with the homogenized emulsion using a high shear rotor-stator type mixer (Omni GLH mixer) at 18,000-24,000 rpm. Triethanolamine was added gradually while mixing until the desired pH and viscosity were achieved.

### Example 2

### Indomethacin 2% Cream

The composition was prepared in accordance with the methodology of Example 1.

TABLE 2

| | Per 100 | Per |
|---|---|---|
| CREAM INGREDIENTS | g cream | 1000 g |
| Lipid Phase | | |
| Indomethacin USP | 2.00 | 20.00 |
| Medium Chain Triglycerides (Labrafac ® CCTG) | 8.00 | 80.00 |
| Egg Lecithin S-75 | 2.00 | 20.00 |
| (±) Camphor USP | 6.00 | 60.00 |
| L-(−)-Menthol USP | 6.00 | 60.00 |
| Tween ™-80 (Polysorbate-80 USP) | 2.00 | 20.00 |
| TPGS (Tocopherol polyethylene glycol 1000 | 0.80 | 8.00 |
| succinate) | | |
| Water Phase | | |
| Sodium Ethylenediaminetetraacetate (EDTA sodium) | 0.10 | 1.00 |
| Bronopol ® (2-Brom-2-nitro-1,3-propanediol) | 0.10 | 1.00 |
| Triethanolamine | 0.50 | 5.00 |
| Ultrez ™ 10 | 0.50 | 5.00 |
| Glycerin | 2.20 | 22.00 |
| Water | 69.80 | 698.00 |

Bronopol® (2-Brom-2-nitro-1,3-propanediol) was added to the water phase as an antibacterial preservative. Ultrez™ was used as a viscosity regulating component instead of Carbopol® without the preliminary hydration step as set forth in Example 1.

### Example 3

### Diclofenac Sodium 1% Cream

The composition of the emulsion for 1% Diclofenac cream presented in Table below. The cream contains approximately 14% of the oil phase with a ratio MCT:Camphor:Menthol of 6:3:4.

TABLE 3

| CREAM INGREDIENTS | Per 100 g cream |
|---|---|
| Medium Chain Triglycerides (Labrafac ® CCTG) | 6.00 |
| (±) Camphor USP | 3.00 |
| L-(−)-Menthol USP | 4.00 |
| Tocopherol succinate | 0.02 |
| Soy Lecithin (Phospholipon ® S-80) | 0.12 |
| Tween ™-80 (Polysorbate - 80) | 2.00 |
| Diclofenac Sodium USP | 1.00 |
| Water | 80.38 |
| Hydrochloric acid 1N | 3.5 |

The oil phase was prepared by dissolving MCT, oil Tocopherol succinate, lecithin, camphor, and menthol at 45° C.

The water phase was prepared by dissolving Diclofenac sodium and Tween™-80 in hot 85° C. purified water.

After mixing the warm oil and hot water phases, hydrochloric acid was added to coarse emulsion while intensive stirring. The pH was adjusted to between 3.5 and 4.2. Homogenization was conducted as described in Example 2. After a fine emulsion was obtained, it was filtered through 0.45 micron PTFE membrane filter. The emulsion was used for cream preparation by addition of Carbopol® 971 as a gelling agent to a final concentration of 1.5% with pH adjustment to between 4.5 and 5.0.

1.5% Diclofenac sodium emulsion (high loading) was prepared by a similar manner. The composition is identical to that tabulated in Table 3. Balance was adjusted with water and hydrochloric acid.

### Example 4

### Ibuprofen 5% Cream

5% Ibuprofen cream was prepared as described in Example 2. The composition of the emulsion for 5% Diclofenac cream is presented in Table 4. The cream contains approximately 26% of the oil phase with a ratio MCT:Camphor:Menthol of approximately 4.25:1:1.

TABLE 4

| | Per 100 | Per |
|---|---|---|
| CREAM INGREDIENTS | g cream | 300 g |
| Ibuprofen | 5.00 | 15.00 |
| Medium Chain Triglycerides (Labrafac ® CCTG) | 13.75 | 41.25 |
| Soy Lecithin (Phospholipon ® S-80) | 1.20 | 3.60 |
| (±) Camphor USP | 3.25 | 9.75 |
| L-(−)-Menthol USP | 3.25 | 9.75 |
| Tween ™-80 (Polysorbate-80, USP) | 2.00 | 6.00 |
| TPGS (Tocopherol polyethylene glycol 1000 | 0.80 | 2.40 |
| succinate) | | |
| Sodium Ethylenediamine tetraacetate (EDTA sodium) | 0.10 | 0.30 |
| Bronopol ™ | 0.10 | 0.30 |
| Triethanolamine | 1.00 | 3.00 |
| Carbopol ® 934P | 1.00 | 3.00 |
| Glycerin | 2.20 | 6.60 |
| Water | 66.35 | 398.10 |

### Example 5

### Piroxicam 2.0% Cream

The composition was prepared by the method described in Example 4, but L-(−)-menthol was replaced with thymol (2-isopropyl-5-methylphenol). The cream contained approximately 28% of the oil phase with a ratio MCT:Camphor:Thymol:Tocopherol acetate of approximately 2:5:5:2.

TABLE 5

| | Per 100 g | |
|---|---|---|
| CREAM INGREDIENTS | cream | Per 250 g |
| Piroxicam | 2.00 | 5.00 |
| Medium Chain Triglycerides (Labrafac ® CCTG) | 3.00 | 7.50 |
| Soy Lecithin (Phospholipon ® S-80) | 2.00 | 5.00 |
| (±) Camphor USP | 7.50 | 18.75 |
| Thymol | 7.50 | 18.75 |
| Tween ™-80 (Polysorbate-80, USP) | 2.50 | 6.25 |
| Tocopherol acetate (Vitamine E acetate) | 2.00 | 5.00 |
| EDTA | 0.10 | 0.25 |
| Carbopol ® 934P | 1.50 | 3.75 |
| Glycerin | 2.20 | 5.50 |
| Triethanolamine | 0.90 | 2.25 |
| Water | 69.70 | 174.25 |

### Example 6

### Reference

Emulsion with Indomethacin, prepared according to U.S. Pat. No. 6,113,921.

TABLE 6

| | Per 100 g cream | |
|---|---|---|
| | 0.5% Indo | 1.0% Indo |
| CREAM INGREDIENTS | (low loading) | (high loading) |
| Indomethacin | 0.5 | 1.0 |
| Medium Chain Triglycerides (MCT oil) | 17.0 | 17.0 |
| Egg Lecithin (Phospholipon ® E-80) | 0.8 | 0.8 |
| Emulphor EL-620 (polyethoxylated | 1.6 | 1.6 |
| castor oil) | | |
| Carbopol ® 940 | 1.7 | 1.7 |
| Glycerin | 2.2 | 2.2 |
| EDTA sodium salt | 0.05 | 0.05 |
| Tocopherol acid succinate | 0.04 | 0.05 |
| Triethanolamine | 0.65 | 0.65 |
| Water | 75.3 | 74.5 |

Indomethacin (0.5 g for low loading and 1.0 g for high loading emulsions) was dissolved in a preheated (60° C.) mixture of egg lecithin, tocopherol succinate and MCT oil. This mixture was emulsified with a water phase (water with Emulfor EL-620, EDTA sodium and glycerin) using a high shear mixer for 5 minutes at 20,000 rpm to form an emulsion.

Further treatment of the emulsion was conducted in a high pressure homogenizer at 800 bar (12,000 psi) for 6 cycles. Thereafter, the emulsion was cooled to room temperature, and pH was adjusted to between 5.6 and 6.5. Part of emulsion was gelled using Carbopol® 940 to form a cream; another part was stored at room temperature in tightly closed amber glass containers for 6 months to observe the physical stability.

Diclofenac sodium (1.0% and 1.5% drug loading) emulsions were prepared in a similar manner and as further described in Example 3.

To estimate drug precipitation, the stored emulsion samples either for the reference Example 6, low and high loaded or invention related (Examples 1 and 2) were filtered through PTFE membrane filters. The Indomethacin content in the filtrates was measured using the HPLC method. FIG. 4 illustrates the results.

Low loaded emulsion, prepared in accordance with U.S. Pat. No. 6,113,921, showed reasonable stability during storage, but with an increase in the initial Indomethacin loading, the final concentration of non-precipitated drug decreases drastically. In contrast, the eutectic mixture vehicle emulsion, prepared in accordance with the present invention, maintains drug content.

Similar results have been obtained for Diclofenac sodium emulsions. In a composition containing 1% of the drug, stability for both formulations was observed. In a composition containing 1.5% Diclofenac sodium emulsion in the menthol-camphor eutectic mixture, stability was observed for at least 3 months at room temperature, while identically loaded reference emulsion demonstrated significant drug precipitation during the same period.

### Example 7

### 2.5% Indomethacin Cream with Acetylated Monoglycerides

To obtain higher loading of Indomethacin in the topical formulation, the oil phase was modified. Medium chain triglycerides were replaced to more polar acetylated triglycerides (Myvacet®, Quest International). Such change in composition allowed to carry out emulsification process without high pressure homogenization, providing stable colloidal composition.

TABLE 7A

| | 2.5% Indomethacin CREAM INGREDIENTS | % |
|---|---|---|
| | OIL PHASE | |
| 1 | Indomethacin USP | 2.50% |
| 2 | Acetylated monoglycerides (Myvacet ®) | 8.00% |
| 3 | Tocopherol acetate synthetic | 2.00% |
| 4 | Camphor | 10.00% |
| 5 | Menthol | 10.00% |
| 6 | Ethoxylated Castor oil | 2.50% |
| 7 | TPGS (d-Alpha Tocopheryl Polyethylene Glycol 1000 | 3.00% |
| | Succinate) | |
| 8 | Vitamin E acid succinate | 0.01% |
| 9 | Lecithin | 1.50% |
| 10 | Ethanol | 1.50% |
| | WATER PHASE | |
| 11 | Water purified | 54.84% |
| 12 | Glycerin | 2.20% |
| 13 | EDTA sodium | 0.10% |
| 14 | Methyl Paraben | 0.40% |
| 15 | Propyl paraben | 0.10% |
| 16 | Carbopol (Ultrez ® 10) | 0.80% |
| 17 | Triethanolamine | 0.55% |
| | pH adjusted to 4.6-5.2 with Triethanolamine | |
| | Total: | 100% |

TABLE 7B

| | 2.5% Indomethacin CREAM INGREDIENTS | % |
|---|---|---|
| | OIL PHASE | |
| 1 | Indomethacin USP | 2.50% |
| 2 | Acetylated monoglycerides (Myvacet ®) | 8.00% |
| 3 | Tocopherol acetate synthetic | 2.00% |
| 4 | Camphor | 10.00% |
| 5 | Menthol | 10.00% |
| 6 | Ethoxylated Castor oil | 2.50% |
| 7 | TPGS (d-Alpha Tocopheryl Polyethylene Glycol 1000 | 3.00% |
| | Succinate) | |
| 8 | Vitamin E acid succinate | 0.01% |
| 9 | Lecithin | 1.50% |
| 10 | Ethanol | 1.50% |
| | WATER PHASE | |
| 11 | Water purified | 55% |
| 12 | Glycerin | 2.20% |
| 13 | EDTA sodium | 0.10% |
| 14 | Bronopol ® (2-Bromo-2-Nitro-1,3-propanediol) | 0.10% |
| 15 | Carbopol (Ultrez ® 10) | 1.0% |
| 16 | Triethanolamine | 0.50% |
| | pH adjusted to 4.6-5.2 with Triethanolamine | |
| | Total: | 100% |

### Example 8

### 2.5% Indomethacin Cream with Liquid Ester Wax

Another hydrophobic compound to be used in 2.5% Indomethacin cream composition is hexadecyloctanoate (cetearyloctanoate “Luvitol EHO from BASF—aliphatic ester, “liquid wax”). This ester has good emolient properties, low irritatioon and high compatibility with cream components.

TABLE 8

| CREAM INGREDIENTS | Per 100 g |
|---|---|
| Indomethacin USP | 2.5 |
| Cetearyl Octanoate (LUVITOL ™ EHO, BASF) | 8.0 |
| Tocopherol acetate synthetic | 2.0 |
| Camphor (racemic) | 10.0 |
| L-Menthol | 10.0 |
| Ethoxylated castor oil | 2.5 |
| TPGS | 3.0 |
| Vitamin E succinate | 0.01 |
| Soya Lecithin | 1.5 |
| Ethanol for lecithin solution | 1.5 |
| Water purified | 55.2 |
| Glycerin | 2.2 |
| EDTA disodium dihydrate | 0.1 |
| Diimidazolydinil urea | 0.1 |
| Carbopol (Ultrez ® 10) | 0.8 |
| Triethanolamine | 0.55 |
| Total: | 100.0 |

### Example 9

### 2.5% Indomethacin Microemulsion with Menthol:Camphor ratio 55:45

TABLE 9

| | Per 100 g |
|---|---|
| Indomethacin USP | 2.5 |
| Menthol | 6.6 |
| Camphor | 5.4 |
| Acetylated monoglycerides | 7.2 |
| Tocopherol acetate | 1.8 |
| Ethoxylated castor oil | 2 |
| TPGS | 2.5 |
| Lecithin S-80 | 1.25 |
| Alcohol | 1.25 |
| Water | To 100 |

### Examples 10 and 11

### Diclofenac Sodium (1.0% and 1.5%) Creams with Acetylated Monoglycerides

Diclofenac emulsions and creams were prepared in a similar manner to Example 7.

TABLE 10

| | Example 10 | Example 11 |
|---|---|---|
| | 1% Diclofenac | 1.5% Diclofenac |
| | Sodium | Sodium |
| Oil phase | | |
| L-Menthol | 6.00% | 6.00% |
| Camphor (racemic) | 6.00% | 6.00% |
| Medium chain triglycerides | 8.00% | |
| Myvacet ® | | 8.00% |
| Tocopherol acetate synthetic | 2.40% | 2.00% |
| Ethoxylated castor oil | 2.00% | 2.40% |
| Ethoxylated lanolin | 3.00% | |
| TPGS | | 2.00% |
| Citric acid anhydrous | 0.20% | 0.30% |
| Lecithin S-80 | 1.20% | 1.50% |
| Ethanol (for lecithin solution) | 1.20% | 1.50% |
| Vitamin E Succinate | 0.01% | 0.01% |
| Water phase | | |
| Diclofenac Sodium | 1.00% | 1.50% |
| Glycerin | 2.20% | 2.20% |
| Water | 65.28% | 64.74% |
| EDTA | 0.10% | 0.10% |
| Bronopol ® | 0.05% | 0.05% |
| Carbopol | 1.00% | 1.20% |
| Triethanolamine | 0.36% | 0.50% |
| TOTAL: | 100.00% | 100.00% |

### Example 12

### 1% Indomethacin Microemulsion Cream

TABLE 11

| | |
|---|---|
| Indomethacin USP | 1.00 |
| Medium chain triglycerides | 5.00 |
| Tocopherol acetate | 0.80 |
| Camphor | 4.00 |
| Menthol | 4.00 |
| Polyethoxylated castor oil | 4.00 |
| Polyethoxylated lanolin | 2.00 |
| Lecithin | 1.00 |
| Vitamin E Succinate | 0.01 |
| Alcohol USP | 1.00 |
| Purified water | 73.95 |
| Glycerin | 2.20 |
| Edetate Disodium | 0.10 |
| Bronopol | 0.10 |
| Carbopol | 0.60 |
| Triethanolamine | 0.24 |
| TOTAL: | 100.00 |

### Example 13

### Ketoprofen 5% Creams with Isopropylmyristate

Ketoprofen formulation cream were prepared in a similar manner to Example 2.

TABLE 12

| CREAM INGREDIENTS | Per 100 g cream |
|---|---|
| KETOPROFEN | 5.00 |
| Isopropylmyristate | 8.00 |
| Labrasol | 3.00 |
| Glycerin monostearate | 0.40 |
| Camphor | 7.50 |
| Menthol | 7.50 |
| Tween-80 | 2.00 |
| TPGS | 0.80 |
| EDTA | 0.10 |
| Bronopol | 0.10 |
| Triethanolamine | 1.00 |
| Carbopol | 1.00 |
| Glycerin | 2.20 |
| Water | 61.40 |

### Examples 14 and 15

### Piroxicam 1% and Meloxicam 1% Creams with Isopropylmyristate

Following creams were prepared in a similar manner to Example 2.

TABLE 13

| CREAM INGREDIENTS Per 100 g | Example 14 | | Example 15 | |
|---|---|---|---|---|
| Piroxicam | 3. | 1.00 | 4. | |
| Meloxicam | 5. | | 6. | 1.00 |
| Acetylated monoglycerides (Myvacet ®) | 7. | 3.00 | 8. | |
| DIPROPYLENE GLYCOL DIBENZOATE | 9. | | 10. | 4.00 |
| (Finsolv ® PG-22) | | | | |
| Soy Lecithin | 11. | 2.00 | 12. | 2.00 |
| Camphor USP | 13. | 7.50 | 14. | 8.00 |
| L-Menthol | 15. | 7.50 | 16. | 8.00 |
| Tyloxapol | 17. | 1.5 | 18. | 1.5 |
| Tween ™-80 (Polysorbate-80, USP) | 19. | 2.50 | 20. | 2.50 |
| Tocopherol acetate (Vitamine E acetate) | 21. | 2.00 | 22. | 1.50 |
| EDTA disodium | 23. | 0.10 | 24. | 0.10 |
| Carbopol ® 934P | 25. | 1.50 | 26. | 1.50 |
| Glycerin | 27. | 2.20 | 28. | 2.20 |
| Triethanolamine | 29. | 0.90 | 30. | 1.0 |
| Water | 31. | 68.20 | 32. | 65.60 |

### Example 16

### Anti Inflammatory Activity “In Vivo”

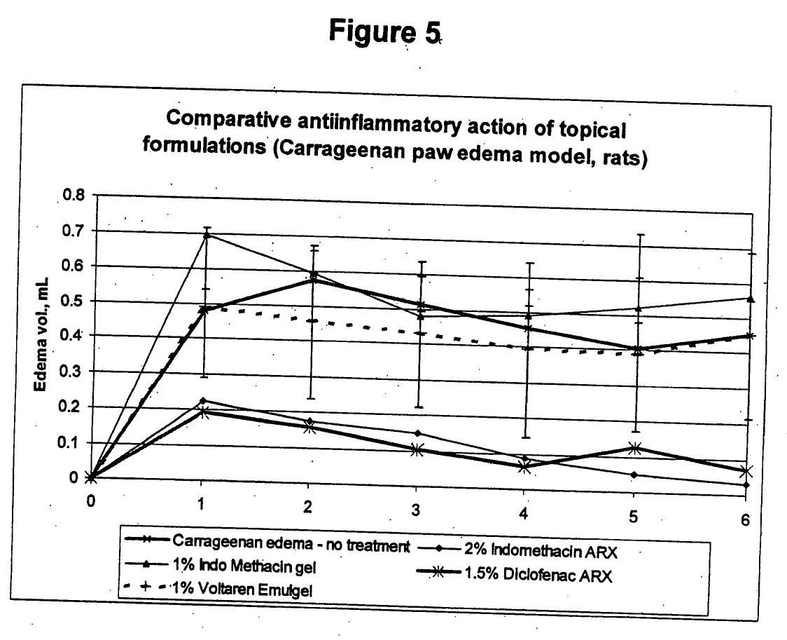

Investigations on animals (rats, carrageenan induced paw edema model) showed significant anti-inflammatory action of the topically applied compositions containing the eutectic vehicle. The results are illustrated in FIG. 5. From a review of FIG. 5, it is evident that the anti-inflammatory efficacy of the formulations of the present invention (ARX) is superior. This is a consequential result of the increased solubility and thus amount of the active ingredient.

TABLE 14
Area Under the Curve (AUC) ratio for edema volume
(calculated by trapezoidal rule for 5 = 0-6 hours).

| GROUP | Ratio, % (± RSD) |
|---|---|
| Control (nontreated) | 100% (±28%) |
| Indomethacin cream (ex. 2) | 32% (±12%) |
| 2 mg of Indomethacin/rat | |
| Indomethacin gel (Methacin ™, Sumitomo Pharm.) | 114% (±39%) |
| 2 mg of Indomethacin/rat | |
| Diclofenac sodium cream (ex. 3) | 30% (±14%) |
| 2 mg of Diclofenac sodium/rat | |
| Voltaren ® Emulgel ® | 82% (±9%) |
| 2 mg of Diclofenac/rat | |
| Salicylic acid trolamin cream (ex. 8) | 65% (±18%) |
| (15 mg of salicylate/rat) | |
| Marketed trolamine Salicylate cream (Aspercream) | 89% (±42%) |
| (15 mg of salicylate/rat) | |

TABLE 15
AUC ratio for edema volume (calculated by trapezoidal rule for
t 0-6 hours) for Diclofenac and Piroxicam formulations

| Relative | | | |
|---|---|---|---|
| AUC ratio | Voltaren | 1.5% Diclofenac | |
| of Edema | Emulgel | cream | |
| volume % | (Novartis) | 1.5 mg of | 2% Piroxicam cream |
| Control | 1.5 mg of | Diclofenac/rat | 2 mg of Piroxicam/rat |
| (nontreated) | Diclofenac/rat | (example 11) | (example 5) |
| 100% | 82% (±33%) | 48% (±19%) | 58% (±24%) |

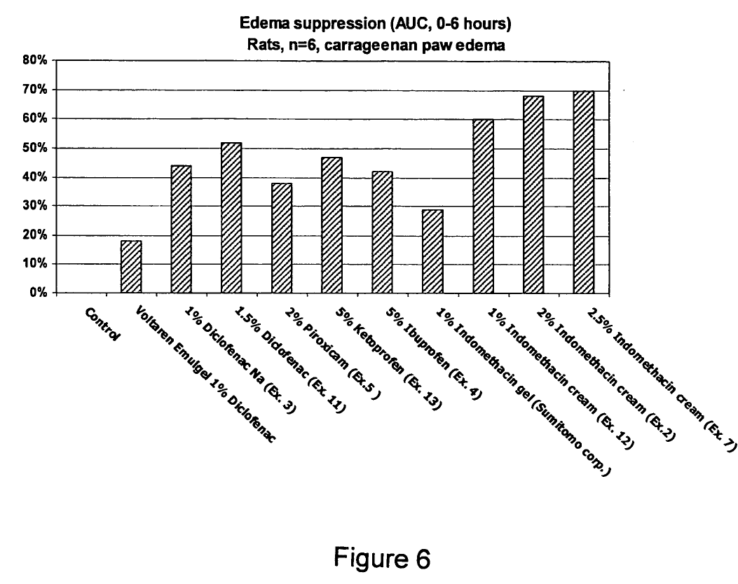

Table 15. Comparative antiinflammatory activity of NSAIDs in different topical formulations (see also FIG. 6).

TABLE 16

| | Edema suppression |
|---|---|
| | (AUC, 0-6 hours) |
| Control | 0% |
| Voltaren Emulgel 1% Diclofenac | 18% |
| 1% Diclofenac Na (Ex. 3) | 44% |
| 1.5% Diclofenac (Ex. 11) | 52% |
| 2% Piroxicam (Ex. 5) | 38% |
| 5% Ketoprofen (Ex. 13) | 47% |
| 5% Ibuprofen (Ex. 4) | 42% |
| 1% Indomethacin gel (Sumitomo corp.) | 29% |
| 1% Indomethacin cream (Ex. 12) | 60% |
| 2% Indomethacin cream (Ex. 2) | 68% |
| 2.5% Indomethacin cream (Ex. 7) | 70% |

### Example 17

### Salicylic Acid Trolamine Cream

TABLE 17

| Component | % |
|---|---|
| Oil Phase | |
| Tocopherol acetate (synth.) | 3.0 |
| Myvacet ™ 9-45K (Quest) | 9.0 |
| L-Menthol (USP) | 2.0 |
| DL-Camphor (USP) | 2.0 |
| Cremophor ® EL (BASF) | 4.0 |
| Supersat ® AWS (RITA) | 2.0 |
| Lecithin (Phosal 53 MCT) | 1.5 |
| Ethyl alcohol | 1.5 |
| Estol ® 1540 (Ethylhexyl Cocoate, | 3.0 |
| Uniquema) | |
| Crotix ® (Croda) | 2.0 |
| Surfhope ® C-1601 (Mitsubishi) | 2.0 |
| Surfhope ® C-1816 (75-25) | 2.0 |
| (Mitsubishi) | |
| Salicylic acid | 7.30 |
| Water phase | |
| Triethanolamine USP/NF | 7.70 |
| Bronopol ™ (BASF) | 0.10 |
| Water purified | 50.90 |
| Total | 100.00 |

### Example 18

### Solubility of Indomethacin and Diclofenac

TABLE 18

| | | Diclofenac | |
|---|---|---|---|
| Solubility at 20° C., mg/g | Indomethacin | (acid) | Piroxicam |
| MCT oil | 4.6 | 8.9 | 0.35 |
| Myvacet 9-45K | 7.03 | 13.2 | 1.4 |
| Tocopherol acetate | 2.51 | 3.1 | 16 |
| Mixed tocopherols | 5.45 | 6.9 | |
| Myvacet + 25% Menthol | 20.08 | 28.3 | |
| Myvacet + 25% Camphor | 13.02 | 15.1 | |
| Menthol-Camphor 1:1 | 159.58 | 105.0 | 2.9 |
| MCT-Menthol-Camphor 4:5:5 | 103.84 | 59.2 | |
| Myvacet-Menthol-Camphor | 57.30 | 64.5 | |
| 4:5:5 | | | |
| Myvacet-Menthol-Camphor- | 57.41 | 67.1 | |
| Tocopherol acetate 4:5:5:1 | | | |
| Isopropylpalmitate | 1.99 | 2.3 | 1.1 |
| Isopropylpalmitate-Menthol- | 56.1 | 51.7 | |
| Camphor 4:5:5 | | | |
| Thymol-Camphor 1:1 | 88.22 | 69.3 | 32 |
| MCT-Thymol-Camphor 2:5:5 | 40.3 | 42.4 | 35 |
| Tocopherol acetate-Thymol- | 36.9 | 36.9 | 28 |
| Camphor 5:3:3 | | | |

### Example 19

### Size Distribution

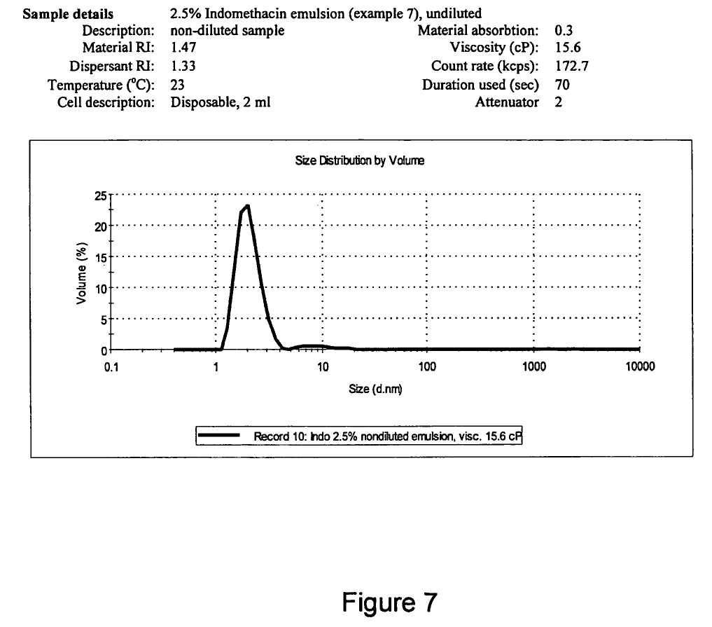

Size distribution of particles in the non-diluted sample of the 2.5% indomethacin emulsion of Example 7 can be seen in FIG. 7. Particle size distribution was measured using Zetasizer particle size analyzer Nano ZS model ZEN3600 (Malvern Instruments Ltd., Grovewood Road, Malvern, Worcestershire WR14 1XZ, UK) with NIBS optic technology, enables measurements of particles from 0.6 nm to 6 microns in non-diluted samples.

Investigated emulsions (not converted to a cream by addition of rheological modifier) were measured undiluted in disposable cell at room temperature (+23° C.). Viscosities of the samples were measured using Brookfield rotational viscometer DV-E in a thermostated vessel at 23±5° C., using LV spindel #1 at 25 rpm. Refractometric indices for oil phases of the emulsions were evaluated using Abbe type optical refractometer (model Mark II, Leica-Reichert, Austria).

Sample of undiluted emulsion (approx. 1 ml) was placed into rectangular polystyrene disposable cell (DTS0012), and particle size distribution was measured. Measurement parameters and settings were entered manually according to manufacturer recommendations. Particle size distribution results are presented by volume (PSD-Volume) distribution

In one embodiment, the non-undiluted solvent vehicle comprise particles in the range of 0.5 to 50 nm. In another embodiment between 1 to 40 nm. In one embodiment, the non-diluted solvent vehicle has particles with an average and/or particle size in said ranges.

While the present invention has been described with reference to what is presently considered to be a preferred embodiment, it is to be understood that the invention is not limited to the disclosed embodiment. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method for solubilizing a non-steroidal anti-inflammatory drug for topical application, comprising: combining the non-steroidal anti-inflammatory drug with a solvent vehicle comprising a liquid eutectic mixture of at least two hydrophobic compounds selected from the group consisting of camphor, menthol and thymol.

2. The method according to claim 1 wherein the solvent vehicle comprises a eutectic mixture comprising camphor and menthol.

3. The method according to claim 2 wherein the camphor and menthol are present in a ratio of 4:1 to 1:4.

4. The method according to claim 3 wherein the camphor and menthol are present in a ratio of 2:1 to 1:2.

5. The method of claim 1 wherein said camphor is selected from the group consisting of D-isomer, L-isomer, racemic camphor or a mixture of camphor isomers.

6. The method of claim 1 wherein said menthol is selected from the group consisting of D-isomer, L-isomer, racemic menthol or a mixture of menthol isomers.

7. The method according to claim 1 wherein the solvent vehicle further comprises a liquid hydrophobic component.

8. The method according to claim 7 wherein the liquid hydrophobic component is selected from the group consisting of pharmaceutically acceptable glycerin ester, aliphatic esters, aromatic esters, waxes, lipids, fats, lipid soluble vitamins, hydrocarbons, silicone polymers, tocopherols, or mixture thereof.

9. The method according to claim 8 wherein said liquid hydrophobic component is tocopherol.

10. The method according to claim 9, wherein the tocopherol is alpha-tocopherol or tocopherol acetate.

11. The method of claim 1, wherein the non-steroidal anti-inflammatory drug is selected from the group consisting of: indomethacin, diclofenac, ketorolac, piroxicam, tenoxicam, ketoprofen, flurbiprofen, ibuprofen, Naproxen, meloxicam and salicylic acid.

12. The method according to claim 11 wherein the non-steroidal anti-inflammatory drug are selected from the group consisting of: diclofenac, piroxicam, indomethacin, ketoprofen, meloxicam, and ibuprofen.

13. The method according to claim 12 wherein the non-steroidal anti-inflammatory drug is diclofenac.

14. The method according to claim 12 wherein the non-steroidal anti-inflammatory drug is piroxicam.

15. The method according to claim 12 wherein the non-steroidal anti-inflammatory drug is indomethacin.

16. The method according to claim 12 wherein the non-steroidal anti-inflammatory drug is ibuprofen.

17. The method of claim 1 further comprising a polar acetylated di or mono-glyceride and/or, a hydrobobic ester.

18. The method of claim 17, wherein the composition further comprises a polar triglyceride.

19. The method of claim 18, wherein the polar triglyceride is selected from the group consisting of medium chain triglyceride and acetylated monoglyceride.

20. A composition for topical application comprising: a non-steroidal anti-inflammatory drug selected from the group consisting of diclofenac, piroxicam, indomethacin, ketoprofen, meloxicam and ibuprofen; and a solvent vehicle for solubilizing said non-steroidal anti-inflammatory drug, said solvent vehicle comprising a liquid eutectic mixture of at least two hydrophobic compounds selected from the group consisting of: camphor, menthol and thymol, and optionally a liquid hydrophobic component.

21. The composition of claim 20 wherein the liquid eutectic mixture comprises camphor and menthol.

22. The composition of claim 21, wherein the camphor and menthol are present in the eutectic mixture in a ratio from 4:1 to 1:4.

23. The composition of claim 21, wherein the ratio of said camphor and menthol are present in said eutectic mixture in a ratio from 2:1 and 1:2.

24. The composition of claim 20 wherein the drug is diclofenac.

25. The composition of claim 20 wherein the drug is piroxicam.

26. The composition of claim 20 wherein the drug is indomethacin.

27. The composition of claim 20 wherein the drug is ibuprofen.

28. The composition of claim 20 wherein said camphor is selected from the group consisting of D-isomer, L-isomer, racemic camphor or a mixture of camphor isomers.

29. The composition of claim 20 wherein said menthol is selected from the group consisting of D-isomer, L-isomer, racemic menthol or a mixture of menthol isomers.

30. The composition of claim 20 wherein the hydrophobic component is tocopherol.

31. The composition of claim 30, wherein the tocopherol is alpha-tocopherol or tocopherol acetate.

32. The composition of claim 20 further comprising a glyceride and/or, a hydrophobic ester.

33. The composition of claim 32, wherein the composition further comprises an acetylated glyceride.

34. The composition of claim 33, wherein the acetylated glyceride is selected from the group consisting of diglycerides and acetylated monoglycerides.

35. The composition of claim 20, wherein said solvent vehicle is evenly distributed in water containing media forming an homogeneous mixture.

36. The composition of claim 35 wherein particles of the solvent vehicle are in the range of 0.5 to 50 nm.

37. The composition of claim 36, wherein particles in said solvent vehicle are 1 to 40 nm.

38. The composition of claim 35 further comprising viscosity modifiers, surfactants, preservatives, fragrances and other excipients to promote stable formulation, suitable for topical application to the skin.
